(19) United States
(12) Patent Application Publication
(10) Pub. No.: US 2015/0011640 A1
(43) Pub. Date: Jan. 8, 2015

(54) GYNECOLOGIC COMPOSITION TO TREAT VULVAR VESTIBULITIS

(71) Applicant: Carlo GHISALBERTI, Sao Paulo (BR)

(72) Inventor: Carlo Ghisalberti, Sao Paulo (BR)

(21) Appl. No.: 14/383,099

(22) PCT Filed: Mar. 5, 2013
(86) PCT No.: PCT/IB2013/000524

§ 371 Date: Sep. 4, 2014

(30) Foreign Application Priority Data
Mar. 8, 2012 (IT) MI2012A000363

(51) Int. Cl. A61K 31/132; A61K 31/167

(52) U.S. Cl. CPC A61K 31/132; A61K 31/167
USPC 514/626; 564/512; 514/674

(57) ABSTRACT

A gynecologic composition contains spermidine to treat vulvar vestibulitis syndrome (VVS). In this therapeutic context, spermidine is effective at around one micromole per applied dose in the form of a simple salt or complex, alone or in combination or in conjunction with auxiliary actives such as anaesthetics and estrogens.

### FIELD OF THE INVENTION

The invention refers to a gynecologic local composition to treat vestibulodynia, best known as vulvar vestibulitis syndrome.

### BACKGROUND

Vulvar vestibulitis syndrome (VVS) is one of the most common causes of genital pain and pain with sexual intercourse, affecting up to 15% of women in fertile age.

This syndrome is characterized by severe pain on vestibular touch or attempted vaginal entry, exquisite tenderness to palpation with a cotton swab, as well as by subtle physical findings confined to vestibular erythema. The etiology of VVS is unknown. Hence, it is probably best to consider VVS as a chronic local inflammatory condition with a wide variety of etiologic causes.

The heterogeneity of possible causative factors in VVS results in multiple treatment regimens into clinical practice (Goldstein A T et al., Vulvodynia: strategies for treatment; Clin Obstet Gynecol. 2005;48(4):769-85). More common prescription refers to local anaesthetics and systemic antidepressant, the latter maybe also for an ancillary inhibitory effect on mast cells degranulation.

Innovation under study includes nifedipine and enoxaparin to improve microvascularity; or botulin, capsaicin, pregabalin, and gabapentin with the aim of reducing the sensibility in the affected tissues.

Scientifically rigorous studies are sorely needed to determine the best approach in VVS. In this frame we have tentatively tested spermidine in sustained delivery system form, which proven effective in in vitro models, as disclosed by co-pending application WO12/017288 and WO12/017290. This therapeutic approach was conceived and is under testing in women's pathologies of dominant atrophic and senescent nature, such as in vaginal atrophy (VA).

While the reparative action performed by spermidine was expectedly beneficial in VA, no response could be initially expected in VVS, a predominantly inflammatory condition with a suspected underlying neurogenic component.

In fact, previous researches disclosed the spermidine pro-inflammaory action. Silva M A in “Role of peripheral polyamines in the development of inflammatory pain” (Biochem Pharmacol. 2011;82(3):269-77) suggested a contribution of Spd and PA in nociception and edema. In this study a local increase in inflammatory sites did correlate to an increase of precursor output, seemingly stimulated by PKC activation. Authors finally stated that limiting PA and Spd level and action could be a method of controlling inflammatory pain.

### SUMMARY

It was surprisingly discovered that spermidine represents an effective medical treatment in vulvar vestibulitis syndrome (VVS).

In an aspect, the invention refers to a local gynecologic composition for the treatment of VVS comprising spermidine, salt, and supramolecular complex thereof.

In another aspect, the invention refers to a local composition for the treatment of VVS comprising spermidine from about 0.1% to 0.001% w/v of the composition.

In a further aspect, the invention refers to the afore-mentioned inventive composition comprising spermidine in combination or in conjunction with one or more anesthetic agent and an estrogenic substance in suitable concentration.

In a yet further aspect, the invention refers to the mentioned inventive composition wherein spermidine is partially or fully substituted with spermine.

These and other features of the invention are best described herein after.

### BRIEF DESCRIPTION OF THE DRAWING

FIG. 1 shows the self-evaluation (subjective score, SS) by weekly questionnaire of a subject with VVS under a 4+4 weeks (W0-W8) treatment protocol with spermidine.

FIG. 2 shows the clinical evaluation (objective score, OS) at baseline (V1), by a next visit after 4 weeks (V2), and by the final visit after the further 4 weeks of treatment.(V3).

### DETAILED DESCRIPTION

The present invention refers to a medicinal, local composition comprising spermidine for the treatment of a gynecologic disorder with prevailing inflammatory and neurogenic signs named Vulvar Vestibulitis Syndrome (VVS).

The active ingredient of the present invention is spermidine, a substance of structure NH₂(CH₂)₃NH(CH₂)₄NH₂, and formula C₇H₁₉N₃.

Spermidine is easily incorporated as salt or complex in water-based gynecologic compositions; or as pure amine in water-free or emulsified gynecologic compositions.

Example of salt form are formed by spermidine with inorganic acid such as HCl, H₂SO₄, H₃BO₃, H₃PO₄, etc., o with organic acid such as acetic, methylsulfonic, ascorbic, lauric, glycolic, lactic, pyruvic, succinic, and citric acid, and the like.

In case of use of spermidine pure amine, partial or full anion exchange may take place in situ in contact with acidic substances in composition. Analogously, a simple salt such as spermidine 3HCl may undergo acid-base exchange within the composition medium. In a further embodiment, the composition of invention will comprise a supramolecular complex with spermidine for the sustained release thereof.

The expression “supramolecular complex” as used herein includes polyacid complex formed by polyanionic polymer(s) and spermidine as well as the inclusion complex of spermidine into cyclodextrins. Technical and operative details are enclosed in co-pending applications WO12/017288 and WO12/017290.

The main inventive object is a topical medicinal composition for treating VVS, wherein “medicinal composition” include drugs, medical devices, and lenitive cosmetics intended for the specific curative purpose.

The inventive composition will comprise spermidine in concentration from about 1% to 0.0001% w/v, more preferably from about 0.1% to 0.001% w/v, even more preferably around 0.01% w/v of the composition.

In the inventive composition, the unitary dose of spermidine will be comprised between 1 mmoles and 1 nmoles/unit dose, preferably between 100 μmoles e 10 nmoles/unit dose, even more preferably around 10 μmoles and 100 nmoles/unit dosea.

The compositions according to the invention, which are administered vaginally, will be presented in semi-fluid forms such as of gel, cream, ointment, etc., or in solid form such as tablets, capsule, pessaries, ovules, etc. for the release of around 1 μmole of spermidine.

Excipients and auxiliary active ingredients to produce a formulation with proper safety, efficacy, patient compliance, aesthetics, acceptability to regulatory authorities, and cost requires are known. E.g., Garg S et al. in Compendium of Pharmaceutical Excipients for Vaginal Formulations Pharmaceutical Technology, Drug Delivery 2001, 14-24; listed vaginal excipients, the functional classification, allowed concentrations (when available), and regulatory status.

The inventive composition may be packaged in ordinary Al or plastic tubes for hand application; or as spray, mousse and other means for the application onto female external genitalia without a direct contact with hand, or other device. In general, no vaginal applicator is needed, unless burdens were widely spread out into the vaginal tract.

In another embodiment, compositions of invention comprise spermidine in combination or in conjunction with an anethetic agent to deliver a fast relief from VVS pain.

Exemplary anesthetic agents include ester-type like benzocaine, chlorprocaine, cocaine, procaine, tetracaine; and amide-type like lidocaine, prilocaine, mepivacaine, bupivacaine, ropivacaine. For use in combination, spermidine and anesthetic agent may be presented in ratio consistent with the desired effect. In particular, the molar ratio of spermidine to anesthetic agent will suitably be approximately 1 to 300. Preferably, this ratio will be between 0.001 to 3 and 1 to 3, and especially from 0.01:3 to 0.1:3.

In another embodiment, compositions of invention comprise spermidine in combination or in conjunction with an estrogenic substance to further improve the therapeutic ratio.

Exemplary estrogenic substances include estrone, estrone esters, estriol esters, estriol, equilin, equilin esters, estradiene, equilenin, ethinyl estradiol, 17β-estradiol, 17β-estradiol esters (i.e. benzoate, cypionate, dienanthate, valerate, etc.), 17α-dihydroequilenin, 17β-dihydroequilenin, 17α-dihydroequilin, 17p-dihydroequilin, 17α-ethynylestradiol, 17α-ethynylestradiol esters, dienestrol, mestranol, mestranol esters, DES, phytoestrogen(s), tibolone, ethynediol and conjugated estrogens.

Conjugated estrogens refer to estrogenic steroidal substances in which one or more functional groups (typically the OH groups) on the steroid exists as a conjugate (typically a sulfate or glucuronide). The conjugated estrogens may be a single conjugated estrogen, or may consist of mixtures of various conjugated estrogens.

For administration in combination, spermidine and the estrogenic substance may be presented in a ratio consistent with the desired effect. In particular, the molar ratio of spermidine to estrogenic substance will suitably be approximately 1 to 1. Preferably, this ratio will be between 0.01 to 1 and 100 to 1, and especially from 0.1:1 to 10:1.

The inventive composition may include other additional active ingredients. Examples include, but not limited to, anti-infective agent such as antibiotics, anti-fungals, antivirals, biocides; calcium antagonists such as nifedipine; heparins such as enoxaparin; botulin; gabaergics such as pregabalin, and gabapentin anti-inflammatories; immuno-suppressant; plant or algal extracts; antihistamines; antioxidants; and a variety of other ingredients such as astringents, fragrances, dyes, vitamins, sunscreens, deodorants, preservatives, and other customary ingredients.

In another embodiment, spermidine is fully or partially replaced by the PA spermine, a substance of structure NH₂(CH₂)₃NH(CH₂)₄NH(CH₂)₃NH₂and formula C₁₀H₂₆N₄.

Other inventive compositions may similarly contain a variety of complementary ingredients as those skilled in the art can select using conventional criteria.

### EXAMPLES

### Example 1

### Test formulation

An investigational device (ID) for local use was prepared in form of gel having ingredients as set forth below.

| Ingredient | amount per 100 ml | |
|---|---|---|
| Spermidine HCl | 10 | mg |
| Sodium hyaluronate | 2.0 | g |
| Sodium benzoate | 0.3 | g |
| Benzyl alcohol | 0.3 | g |
| Phenoxyethanol | 0.5 | g |
| Citric acid | q.b. to pH 5.35 | |
| Purified water | q.b. to 100 | ml |

The resulting transparent gel was packaged in 30 ml Al tube in a GMP/GLP facility.

### Example 1

### Pilot Study on VVS

The general design of clinical protocol is illustrated herein after, with a summary of the key protocol chapter given in brief.

### Primary Outcome Measures: Efficavy

Change in physical signs of vestibulitis on physical exam after 4+4 weeks of treatment by gynaecology visit ranked with a vulvoVaginal Health Index (vVHI), whose 1-to-5 score is illustrated in Table I.

TABLE I
vVHI by Objective Score (OS)

| | SCORE | | | | |
|---|---|---|---|---|---|
| | 1 | 2 | 3 | 4 | 5 |
| Vulvar inflammation | Vivid red | Very red and | Intermediate, | Mostly | Rosy and |
| (redness) | with rashes | inflammed | minor spots | rose- | healthy |
| | | | | colored | |
| Musculature contraction | Highly overal | Anus or | Intermediate | Fair-to- | Excellent, |
| | contracted | pelvis | | good | fully |
| | | contracted | | | relaxed |
| Pain at speculum | Very strong | Strong | Moderate | Minimal | None |
| insertion | | | | | |
| Epithelial integrity | Petechiae, | Bleeds with | Not friable, | Almost | Normal |
| (mucosa) | ulcerate | ligh contact | thin | fully | |
| | mucosa | | epithelium | trophic | |

Change in self-reported, subjective symptoms (SS) of VVS from baseline to 4+4 weeks by weekly questionnaire, whose 0-to-3 score is illustrated in Table II.

TABLE II
Subjective score (SS)

SCORE

| | 0 | 1 | 2 | 3 |
|---|---|---|---|---|
| Pruritus (Hitching) | None | Mild | Moderate | Severe |
| Burning | None | Mild | Moderate | Severe |
| Dryness | None | Mild | Moderate | Severe |
| Dyspareunia | None | Mildt | Moderate | Severe |

Meaning
0 = none (no symptoms during 24 hours);
1 = light (tolerable symptoms in few, short episode during 24 hours);
2 = moderate (tolerable symptoms for most 24 hours, with limited impairment of daily tasks);
3 = severe (hardly tolerable symptoms for most 24 hours, with relevant impairment of daily tasks).

### Secondary Endpoint: Tolerability and Safety

Tolerability, scored from excellent to poor, is included on patient's questionnaire. Safety was monitored from the adverse events (AE) report, that were recorded at all visits from subject experience or by physician controlled, to be reported and described in full length referring to date of beginning, intensity and duration.

### Inclusion Criteria

Main inclusion criteria consist of:
signature of the informed consensus;
women complaining a typical VVS, thereby confirmed by a local gynaecologic visit;
collaborative subjects who are able to follow instruction and protocol schedule.

### Exclusion Criteria

subjects unable to supply a valid informed consensus;
subjects using estrogens, or combined estrogens and progestinics;
presence of autoimmune diseases, allergies, diabetes and hypertension;
presence di cancer or chronic disease with less-then 2 year of life expectancy;
therapy with corticosteroids or immuno-modulators within the past 3 weeks;
previous or concomitant vulvovaginal laser therapy o crio-therapy;
subjects unable to follow the protocol or comprehend scopes and effects;
subjects under whatever experimental drug within the past 8 weeks.

### General Design of the Study

Visit 1 (V1, week 0): Enrolment with baseline assessment, ID delivery and instruction to start treatment by 3 applications per week;

Visit 2 (V2, week 4): After 4 weeks of treatment, objective assessment and next ID delivery for a new cycle with indication of two applications per week;

Visit 3 (V3, week 8): End of 4+4 weeks of treatment with final objective assessment and delivery of the questionnaire.

### Doses and Treatment Regimen

IP was delivered at V1 and V2 with instruction to apply on vulvar area by hand 3 times per week during the first 4 weeks; followed by 3 times per week during next 4 weeks.

### Example 3

### Typical Case Report

A Caucasian woman aged 38 presented to the ambulatory asking medication for recalcitrant VVS. Beside that, health conditions were good, with no sign of allergy, sensitization, nor immuno-reactivity. She was primipara with regular menstrual cycles.

History Vulvodynia started 5 years before. Past attempts to treat VVS were various with no significant results. She furthermore reported recurrent vulvovaginitis from candidosis. Patient performed a pap test within past 6 month with negative response. She accepted to enter the pilot study after signing the informed consensus.

First visit (V1) Upon external exploration, vulva displayed reddish vulvar vestibulus as well as redness on inner labia minora. By inserting speculum, patient experienced strong pain and contracted perineum muscles. Instead, inner vaginal mucosa appeared normal. On SS, patient scored a fair vaginal burning, moderate itching and severe dyspareunia.

Second visit (V2) After 4 weeks of treatment by 3 weekly applications the patient referred about the increase of itching and burning by week 6, then treated with local anti-fungal. Mucosa looked pink with reduced reddish areas. Speculum insert provoked less pain. Patient avoided sexual intercourses, hence dyspareunia was scored as unchanged.

Third visit (V3) After second cycle of 4 weeks by 2 weekly applications, examination revealed an almost normalized vulvar mucosa. Patient did not complain nor perceive pain at speculum insertion. During this period an episode of candidosis were medicated with local antibiotic therapy. Dyspareunia was scored as moderate.

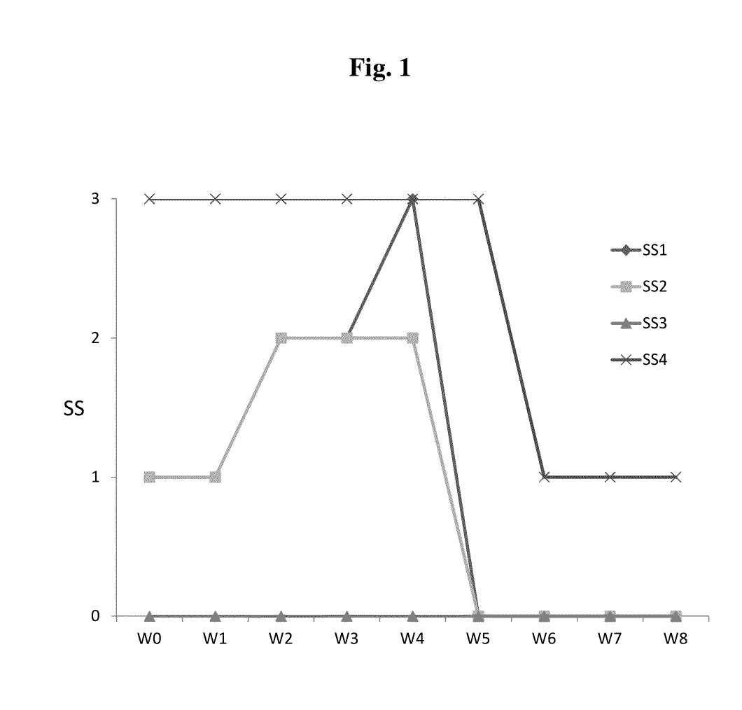

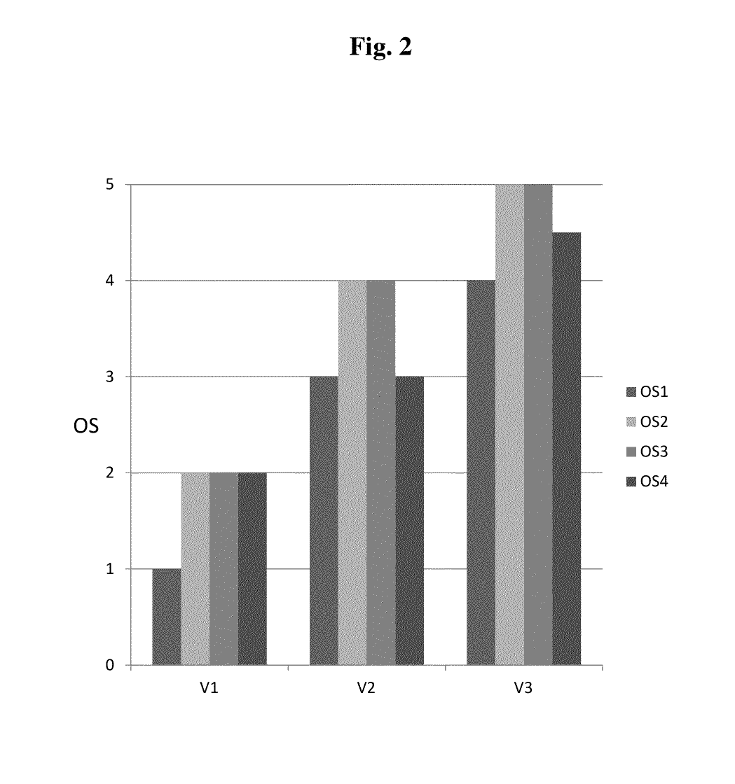

Conclusion Clinical remission was finally attained, as denoted by objective scores (SS) and objective scores (OS) trends, which are plotted in FIG. 1 and FIG. 2, respectively. Interestingly, the improvement already found at visit 2 (V2) by objective examination did not correspond to the perceived symptoms, which persist or even worsened (beside the transient burdens due to candidosis in W4-W5) until a sudden improvement noticed from W6 on. Nonetheless, the two scores converged at the end of the treatment.

### Example 4

### Combination Cream with Anesthetic

An emulsion was prepared by mixing under turbo-emulsifier the oily phase melted at around 70° C. with the water-soluble warmed at the same temperature, which end up with a cream having ingredients as set forth below.

| | |
|---|---|
| Ingredient | amount per 100 ml |

Oily phase

| | | |
|---|---|---|
| Cetyl alcohol | 5.0 | g |
| Glyceryl stearate | 2.5 | g |
| PEG100 stearate | 2.5 | g |
| Dicaprylyl ether | 5.0 | g |
| Cyclopentaxiloxane | 5.0 | g |
| Lidocaine (base) | 2.0 | g |

Water phase

| | | |
|---|---|---|
| Spermidine HCl | 10 | mg |
| Methylisoxazolidone, 10% sol. | 0.1 | g |
| Benzyl alcohol | 0.3 | g |
| Purified water | q.b. to 100 | ml |

The resulting cream was packaged in 30 ml Al tube under GMP/GLP provisions. It provides an immediate pain relief from the very beginning of the spermidine therapy.

### Example 5

### Combination gel with anesthetic

The emulsion obtained in Example 1 was modified by adding 10 mg g of b-estradiol instead of 2 g of lidocaine.

The innovation entails the local use of spermidine to treat VVS. It should be understood that the foregoing relates only to preferred embodiments and to applicative examples of the present invention and that numerous modifications or alterations may be made therein without departing from the spirit and the scope of the invention as set forth in the appended claims.

1. A gynecologic composition for local application comprising spermidine to treat vulvar vestibulitis syndrome (VVS).

2. The gynecologic composition for local application according to claim 1 wherein the spermidine is present as a salt in a concentration between 100 μmoles and 10 nmoles per unit dose.

3. The gynecologic composition for local application according to claim 1 wherein the spermidine is present within a supramolecular complex in a concentration between 100 μmoles and 10 nmoles per unit dose.

4. The gynecologic composition according to claim 2 wherein the spermidine is present in a concentration between 10 and 0.1 μmoles per unit dose and the composition further comprises at least one physiologically acceptable excipient.

5. The gynecologic composition for local application according to claim 1 in the form of a gel, a cream, an ovule, a solution, a tablet, or other another form suitable for local application to a vulva.

6. The gynecologic composition for local application according to claim 4 further comprising an additional active ingredient.

7. The gynecologic composition for local application according to claim 1 further comprising an anaesthetic agent.

8. The gynecologic composition for local application according to claim 1 further comprising an estrogenic substance.

9. The gynecologic composition for local application according to claim 1 further comprising spermine.

10. The gynecologic composition according to claim 3 wherein the spermidine is present in a concentration between 10 and 0.1 μmoles per unit dose and the composition further comprises at least one physiologically acceptable excipient.

11. A gynecologic composition for local application according to claim 2 in the form of a gel, a cream, an ovule, a solution, a tablet, or other another form suitable for local application to a vulva.

12. The gynecologic composition for local application according to claim 3 in the form of a gel, a cream, an ovule, a solution, a tablet, or other another form suitable for local application to a vulva.

13. The gynecologic composition for local application according to claim 4 in the form of a gel, a cream, an ovule, a solution, a tablet, or other another form suitable for local application to a vulva.

14. The gynecologic composition for local application according to claim 2 further comprising an anaesthetic agent.

15. The gynecologic composition for local application according to claim 2 further comprising an estrogenic substance.

16. The gynecologic composition for local application according to claim 3 further comprising an anaesthetic agent.

17. The gynecologic composition for local application according to claim 3 further comprising an estrogenic substance.

18. The gynecologic composition for local application according to claim 4 further comprising an anaesthetic agent.

19. The gynecologic composition for local application according to claim 4 further comprising an estrogenic substance.

20. The gynecologic composition for local application according to claim 9 further comprising an anaesthetic agent.

21. The gynecologic composition for local application according to claim 9 further comprising an estrogenic substance.

22. A gynecologic composition for local application comprising spermine to treat vulvar vestibulitis syndrome (VVS).

23. The gynecologic composition for local application according to claim 22 further comprising an anaesthetic agent.

24. The gynecologic composition for local application according to claim 22 further comprising an estrogenic substance.
